(19) United States
(12) Patent Application Publication
(10) Pub. No.: US 2004/0082855 A1
(43) Pub. Date: Apr. 29, 2004

(54) Tumor dose enhancement using modified photon beams and contrast media

(75) Inventors: James Robar, Halifax (CA); Monty A. Martin, Vancouver (CA); Silvia A. Riccio, North Vancouver (CA)

Correspondence Address: OYEN, WIGGS, GREEN & MUTALA, 480 - THE STATION, 601 WEST CORDOVA STREET, VANCOUVER, BC, V6B 1G1, CA

(73) Assignee: BC Cancer Agency, Vancouver (CA)

(21) Appl. No.: 10/621,575

(22) Filed: Jul. 18, 2003

Related U.S. Application Data
(60) Provisional application No. 60/396,714, filed on Jul. 19, 2002.

(51) Int. Cl. A61B 6/00

(52) U.S. Cl. 600/436

(57) ABSTRACT

A method of radiotherapy includes irradiating a treatment volume containing a high atomic number contrast medium with a photon beam characterized by a peak energy of at least 1 MeV and a mean energy in excess of 250 keV. The irradiation may be provided by a beam from a linear accelerator operating without a flattening filter. The linear accelerator may have multiple modes including one or more modes for operating with a flattening filter and one or modes for operating without a flattening filter.

### CROSS REFERENCE TO RELATED APPLICATION

This application claims the benefit of the filing date of application Ser. No. 60/396,714 filed on 19 Jul. 2002, which is hereby incorporated herein by reference in its entirety.

### TECHNICAL FIELD

The invention relates to radiotherapy.

### BACKGROUND

A principle goal of radiotherapy (which includes radiosurgery) is to deliver a desired well-defined dose of radiation to a treatment volume within a subject. The volume may, for example, be the volume of a malignant or benign tumor. At the same time it is desirable to minimize the dose delivered to surrounding tissues so as to spare the surrounding tissues from radiation-induced damage. This is especially important in cases where the treatment volume is closely adjacent to structures which are susceptible to radiation-induced damage such as the brainstem.

It is known to shape radiation beams using, for example, multi-leaf collimators, so that the radiation beam has a shape which closely matches the projection of the treatment volume at the angle of incidence of the radiation beam. Even when shaped beams are used it is not possible to completely avoid irradiating tissues surrounding the treatment volume because:

there is a limit to the precision with which the beam can be shaped;

the subject may move during treatment or between treatment fractions;

the organ containing the treatment volume may shift within the subject during treatment or between treatment fractions; and,

there may be misalignment of the subject with the radiation source.

Iwamoto et al. Radiation dose enhancement therapy with iodine in rabbit VX-2 brain tumors Radiother, Oncol, 8, 161-170 (1987); Mello R Set al. Radiation dose enhancement in tumors with iodine Med. Phys. 10 75-8 (1983) and Norman A, et al. Iodinated contrast agents for brain tumor localization and radiation dose enhancement Invest. Radiol. 26 S120-21 (1991) propose a technique which has become known as “phototherapy” or “CTRx” for enhancing the dose delivered to a tumor. Phototherapy involves the use of a contrast medium in combination with low energy x-ray beams to increase the radiation dose delivered to the treatment volume relative to the dose delivered to surrounding tissues. The contrast medium is selectively present in the treatment volume and increases the atomic cross section for photoelectric absorption of the x-ray beam in the treatment volume. Rose J H et al. First experience with radiation therapy of small brain tumors delivered by a computerized tomography scanner Int. J. Radiat. Oncol. Biol. Phys. 30 24-5 (1994) used a contrast medium and a modified CT scanner to treat human brain metastases.

While it has been shown that phototherapy can enhance the radiation dose delivered to a tumor, phototherapy has several significant limitations. These include:

The low energy x-ray beams used in phototherapy cause very high doses to be delivered to the skull and other superficial tissues, even when multiple beams are used. This problem is exacerbated because the maximum dose for each beam occurs at the surface and bone has a greater effective atomic number (approximately 12.3) relative to tissue (approximately 7.5). The inadvertent dose enhancement in bone has been shown to persist even for multiple arcs of a 140 kVp beam, for example (Mesa et al 1999).

For high levels of dose enhancement, uniformity within the treatment volume can be poor due to uneven attenuation of the beam by overlying tissues (for tumors that are not centrally located) and attenuation of the beam within the treatment volume itself (Mesa et al 1999).

It can be complicated and expensive to provide a modified CT machine or other device suited for use in delivering phototherapy.

U.S. Pat. No. 6,125,295 entitled Pharmaceutically enhanced low-energy radiosurgery discloses the use of x-rays generated from low-energy orthovoltage x-ray sources to irradiate tumors containing contrast agents. Orthovoltage X-ray sources typically provide peak energies of (140-250 kVp) and average photon energies significantly less than the peak energies.

Linear accelerators providing higher energy photon beams are used for radiotherapy. Photons having energies in the MeV range are not effectively absorbed by available contrast media.

Despite the progress that has been made in the application of radiotherapy and radiosurgery, there remains a need for improved methods for delivering doses of radiation to treatment volumes while minimizing the dose delivered to surrounding tissues.

### SUMMARY OF THE INVENTION

One aspect of this invention provides methods which may be applied for delivering a radiation dose to a treatment volume in a subject. The methods involve providing a high atomic number contrast medium (i.e. a contrast medium having atoms with an atomic number of 50 or more) in the treatment volume and directing a beam of radiation at the treatment volume wherein the beam of radiation has an energy spectrum profile having a peak energy in excess of 500 KeV, and preferably in excess of 1 MeV. The beam has a mean energy in excess of 250 KeV. The beam has a significant number of photons which have energies low enough to interact with the contrast medium. In specific embodiments the radiation beam is generated by a linear accelerator having no flattening filter. The linear accelerator may, for example, be set to generate x-rays having maximum energies in the range of 1 MeV to 20 MeV, preferably in the range of 1 MeV to 5 MeV and most preferably in the range of 1 MeV to 4 MeV.

Another aspect of the invention provides a linear accelerator for use in radiotherapy, the linear accelerator comprising:

an electron accelerator;

a target disposed to receive a beam of electrons from the electron accelerator to generate a photon beam;

one or more flattening filters selectively insertable into a path of the photon beam;

a controller;

an interlock mechanism connected to signal to the controller when one of the one or more flattening filters is in the photon beam and when none of the one or more flattening filters is in the photon beam;

the linear accelerator having a first operating mode into which the controller can place the linear accelerator when the interlock mechanism indicates that the flattening filter is in the path of the photon beam and a second operating mode into which the controller can place the linear accelerator when the interlock mechanism indicates that one of the one or more flattening filters is in the path of the photon beam;

wherein, when the linear accelerator is in the first operating mode, the controller causes the electron beam to be operated at a reduced voltage in the range of 1 MV to 4 MV to produce the photon beam having an energy spectrum characterized by a maximum photon energy in excess of 1 MeV and a mean photon energy in excess of 250 keV and when the linear accelerator is in the second operating mode the controller causes the electron beam to be operated at a voltage in excess of the reduced voltage.

Further aspects of the invention and features of specific embodiments of the invention are described below.

### BRIEF DESCRIPTION OF THE DRAWINGS

In drawings which illustrate non-limiting embodiments of the invention,

FIG. 1 is a block diagram of a prior art linear accelerator;

FIG. 2 is a plot of normalized fluence as a function of energy for a number of photon beams;

FIG. 3 is a plot showing measured and calculated dose as a function of depth;

FIG. 4 is a schematic perspective illustration of a phantom model used in a simulation to test example embodiments of the invention;

FIGS. 5A, 5B and 5C are plots showing calculated dose as a function of depth for various contrast media concentrations;

FIGS. 6A and 6B are plots showing calculated dose as a function of depth for various beam qualities and various contrast media concentrations respectively;

FIG. 7A is a plot of dose enhancement factor as a function of iodine contrast medium concentration for various photon beams;

FIG. 7B is a plot of dose enhancement factor as a function of gadolinium contrast medium concentration for various photon beams;

FIG. 8 is a plot of relative dose as a function of depth for a simulated tumor wherein contrast medium concentration is greater in outer regions of the tumor than it is in inner regions of the tumor; and,

FIG. 9 is a block diagram illustrating the construction of a multi-mode linear accelerator according to an embodiment of the invention.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

This invention provides radiotherapy and radiosurgery methods which provide dose enhancement within one or more treatment volumes in a subject. The method involves providing a contrast medium in the treatment volume(s) and irradiating the treatment volumes with radiation having an energy spectrum as described herein. Various contrast media can be used. The contrast media contain atoms of one or more elements having high atomic numbers (i.e. atomic numbers Z in excess of 50). The contrast media may contain, for example one or more of iodine (Z=53), gadolinium (Z=64), lutetium (Z=71) and gold (Z=79). Mesa et al. Dose distributions using kilovoltage x-ray and dose enhancement from iodine contrast agents Phys. Med. Biol. 44 1955-68 (1999) describes some suitable contrast media. Greater dose enhancement can be achieved by using contrast media having higher effective atomic numbers.

Gd-DPTA is generally preferable to iodine-based contrast media. Compared to iodinated, non-ionic x-ray contrast medium, Gd-DTPA introduces a comparatively small amount of osmotically active particles into the body and thus offers improved tolerability. While the spectrum of adverse effects is similar for non-ionic iodine contrast medium and Gd-DTPA, the frequency of adverse reaction is two to three times lower for the latter.

The contrast media may be introduced into the treatment volume so that the treatment volume retains the desired contrast medium concentration throughout irradiation in any suitable way. Where the treatment volume is made up of a tissue that preferentially takes up a contrast medium, the contrast medium may be injected intravenously or intra-arterially. Injected contrast media tend to localize in tumors of the central nervous system, cranial nerves and dura due to a variety of mechanisms which include hypervascularity and damage to the blood brain barrier caused by invasive tumor growth. Iodine-based contrast media of the type used to enhance contrast in computed tomography (CT) imaging are incorporated preferentially in the volume of a variety of tumors including acoustic neuromas, meningiomas, metastases, pituitary adenomas, astrocytomas and craniopharyngiomas. Similarly, gadolinium-based contrast media of the type are used in magnetic resonance imaging (MRI) to provide selective image enhancement of tumors relative to the surrounding anatomy are preferentially taken up by certain types of tumor.

Contrast media may also be introduced into a treatment volume by direct injection through any suitable catheter. This is particularly practical in extra-cranial lesions. Maintaining a desired contrast medium concentration in a tumor by using a bolus injection followed by slow infusion is an example of this method of introducing a contrast medium into a treatment volume.

In addition to widening the range of lesions for which dose enhancement is possible, direct administration may permit higher concentrations of the high-Z contrast medium atoms to be realized in the treatment volume, thereby increasing levels of dose enhancement. Extra-cranial indications for which dose enhancement may be particularly useful include lesions proximal to critical organs or tissues. For example, tumors such as para-spinal metastases would be suitable candidates for dose enhancement since improved sparing of the adjacent spinal cord may be possible.

In selected cases the take up of contrast medium in the treatment volume may be enhanced by modification of blood-brain barrier permeability, or increasing permeability of blood vessels as described by Norman et al. X-ray phototherapy for solid tumors Acad. Radiol. 5 S177-9 (1998).

The contrast medium is selected and introduced in such a manner that it is not present in unacceptably large amounts within adjacent structures during irradiation. Ideally the concentration of contrast medium within the treatment volume is uniform. For many brain lesions, there are known contrast media for which the uptake in normal tissues is minimal. Studies of Gd-DTPA uptake in extraaxial lesions and normal structures have shown lack of enhancement of both grey and white matter. Veins, dural sinuses and choroid plexus do exhibit early enhancement, but this has been shown to wash out rapidly.

Pituitary tumors are typically not suited to treatment with Gd-DTPA contrast medium dose enhancement, since both the pituitary stalk and gland exhibit gadoliniumenhancement during MRI.

For many tumor types, the contrast medium concentration will vary spatially with the degree of blood-brain barrier disruption and neovascularity. This, in turn, can cause spatial variation of dose enhancement within the tumor volume.

The presence of a contrast medium within a treatment volume increases the effective atomic number of the treatment volume relative to the surrounding tissues. For certain photon beam qualities, this increase in effective atomic number results in a change of photon interaction cross-section within the treatment volume.

X-rays can interact with matter in several ways including by photoelectric absorption, elastic scattering and Compton scattering. Low energy x-rays (e.g. x-rays having energies not exceeding about 400 keV) interact with contrast media in a treatment volume predominantly by the photoelectric absorption interaction. The energy range at which photons interact by the photoelectric absorption mechanism may be called a photoelectric absorption range.

The relationship between the atomic cross section for photoelectric absorption and the atomic number exhibits a Z⁴ to Z^{4.8} dependence. Therefore, by increasing the effective atomic number within the treatment volume, photon absorption is augmented within the treatment volume for photons having energies in the photoelectric absorption range.

It is common to use photon beams having peak energies of several MeV in radiotherapy. Linear accelerators capable of producing such photon beams are widely used. However, the vast majority of photons in the spectrum of the photon beams produced by a linear accelerator operating at MeV energies have energies for which Compton scattering is the predominant interaction in both tissue and tissue loaded with contrast medium. Contrast media provide no significant dose enhancement in such beams. For example, Mello et al. (above) disclose that there is no significant dose enhancement when a Cobalt-60 beam is used to irradiate human lymphocytes after absorption of iodine.

The inventors have discovered that it is possible to achieve dose enhancement in contrast medium-containing treatment volumes by providing megavoltage irradiation from a linear accelerator with the flattening filter removed. This can be achieved while significantly reducing the radiation dose delivered to overlying tissues in comparison to methods which use lower energy x-rays generated by orthovoltage sources. Where the treatment volume is within a subject's skull the methods of the invention may be used to spare the subject's scalp and skull from the excessive radiation doses that occur in photovoltage therapies.

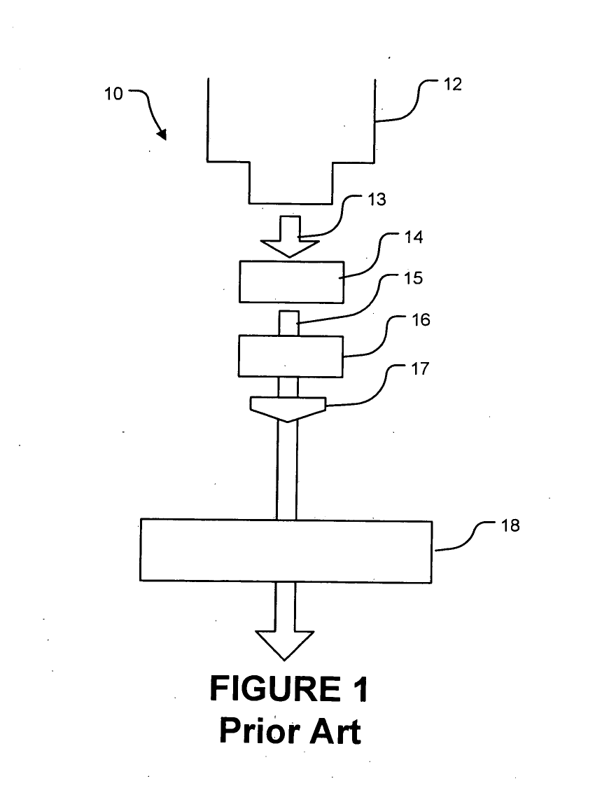

FIG. 1 is a schematic view of the radiation path of a typical linear accelerator 10. Accelerator 10 has an electron accelerator 12 which generates an electron beam 13 in which electrons have an energy in the MeV range. Electron beam 13 interacts with a target 14. Target 14 typically comprises a tungsten disk. The interaction between electron beam 13 and target 14 generates an X-ray beam 15. X-ray beam 15 passes through a primary collimator 16, a flattening filter 17 and a multi-leaf collimator 18 before being directed at a subject to be treated.

The purpose of flattening filter 17 is to make X-ray beam 15 have an intensity which is substantially uniform across the field at a desired location. Beam 15 is typically more intense in its central part and less intense at its edges. Flattening filter 17 attenuates X-ray beam 15 more in its central part and less at its edges to achieve substantial uniformity. Flattening filter 17 is typically made of metal and is often made of a low atomic number metal such as aluminum. A linear accelerator may have different flattening filters for use at different energies.

A flattening filter can significantly affect the overall output of a linear accelerator. To protect subjects from inadvertent overexposure to radiation, when a new linear accelerator is commissioned its radiation output is characterized. This is done with a specific flattening filter in place. If the flattening filter is changed then the linear accelerator should be recommssioned. Most medical linear accelerators are always operated with a flattening filter in place.

The inventors have determined that, with no flattening filter in place, a medical linear accelerator operating in the MeV range produces a photon beam having an energy spectrum such that significant dose enhancements can be obtained in contrast-medium-containing treatment volumes without delivering large doses to the skin and other superficial tissues as occurs in prior phototherapy methods.

A flattening filter tends to preferentially filter out lower energy photons. It has been shown through Monte Carlo simulation and analytic calculation of bremsstrahlung spectra using the thin-target Schiff expression that the removal of flattening filtration significantly increases the relative proportion of photons in the low energy region (see for example, Sixel and Faddegon Calculation of x-ray spectra for radiosurgical beams Med. Phys. 22 1657-61 (1995)). For a 6 MV radiosurgical beam, for example, the peak of the photon energy distribution is shifted from approximately 1.0 MeV to approximately 0.3 MeV by removing the flattening filter. Although this increases the relative proportion of low-energy photons in the incident spectrum, the mean photon energy remains in the megavoltage range.

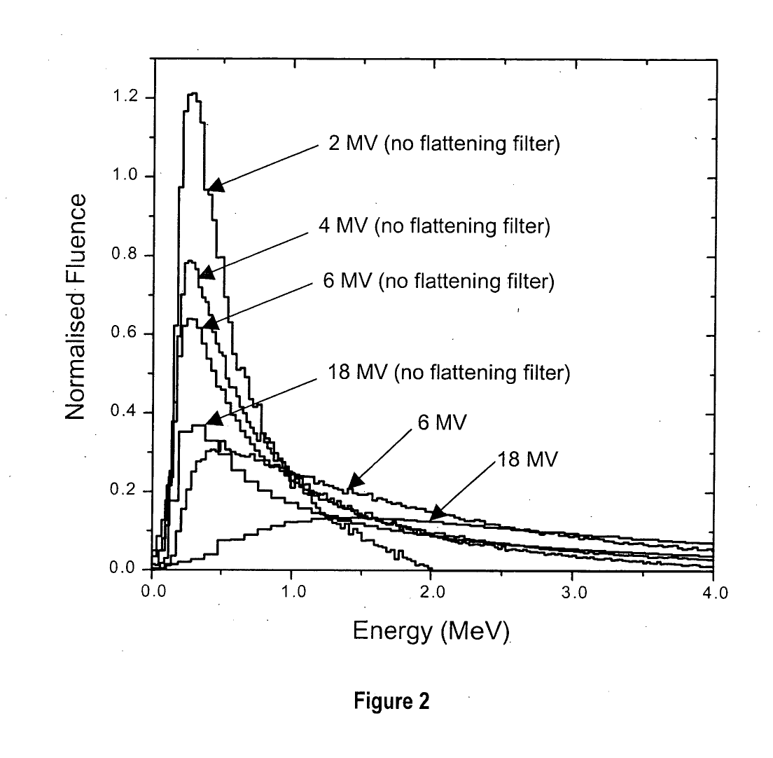

FIG. 2 is a plot of energy spectra for a number of photon beams produced by a linear accelerator with and without a flattening filter in place. The curves are normalized to have equal area. Each curve traces the relationship between photon energy and fluence. Comparison of either the 6 MV or 18 MV spectra with and without flattening filtration clearly demonstrates the significant, relative increase in the photon population below 1 MeV in the beam which lacks flattening filtration. This results in a decrease of mean photon energy, for example, from 1.9 MeV to 1.3 MeV and from 4.6 MeV to 3.4 MeV for the 6 MV and 18 MV beams, respectively.

It has been found that dose enhancement can be obtained in treatment volumes containing high atomic number contrast media by irradiation with a photon beam having an energy spectrum wherein a maximum photon energy is in excess of 1 MeV and a mean photon energy is in excess of 250 keV, preferably in excess of 1 MeV. The use of such a photon beam significantly reduces (as compared to phototherapy) the dose delivered by the beam to skin and other superficial tissues.

### EXAMPLE

Monte Carlo modeling of a linear accelerator (Varian™ model 2100 C D) was done using BEAM 2000™ software running in a distributed fashion on a generic network queuing system (GNQS) cluster of ten Pentium II™ computers. The linear accelerator model was specified using dimensions and materials provided by the manufacturer (Varian Oncology, Palo Alto, Calif.) equipped with a a 25 mm diameter stereotactic collimator (BrainLAB, AG, Heimstetten, Germany). The jaws of the collimator were set to 5 cm×5 cm at isocentre.

Phase-space files containing the energy, position, charge and angle of incidence of each particle were recorded in a plane perpendicular to the central axis and immediately below the stereotactic collimator. 6 MV and 18 MV beams were modelled with the appropriate flattening filters. The mean energy of the electron beam incident upon the target was determined with reference to the bending magnet shunt current on the actual treatment unit. This method of selecting the electron beam energy was effective in producing beam models which agree well with measured depth dose characteristics in water.

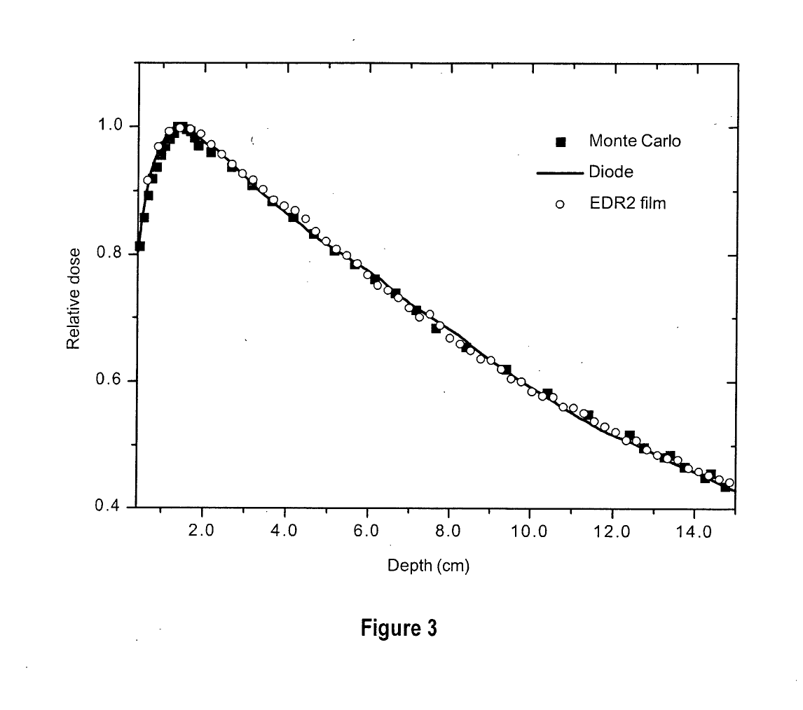

FIG. 3, for example, shows a comparison of Monte Carlo calculated depth dose in water with that obtained using a silicon diode dosimeter (Scanditronix, Uppsala, Sweden) and EDR2 radiographic film (Eastman Kodak Company) for the 6 MV 25 mm diameter beam. The film measurement and calibration were performed as described previously Robar and Clark, The use of radiographic film for linear accelerator stereotactic radiosurgical dosimetry, Med. Phys. 26, 2144-50 (1999).

Additional simulations were also conducted after removing the flattening filter from the model for 2 MV, 4 MV, 6 MV and 18 MV beams. At least 200M histories were followed for each simulation to create phase-space files with sufficient particles for subsequent simulation of patient irradiation (see below).

To examine the degree to which dose enhancement is produced with high-quality photon beams, the 18 MV phase-space file of the photon beam (modeled as described above) was used, in addition to a 24 MV published spectrum (Mohan et al 1985). For comparison purposes, published 10 MV and 15 MV spectra, Mohan et al. Energy and angular distributions of photons from medical linear accelerators, Med. Phys. 12, 592-7 (1985), were also used. Simulations with a 250 kVp incident spectrum were also performed to allow comparison of results to values of dose enhancement in phototherapy.

Each spectrum was used as input to a simulation to model the irradiation of a human head containing a tumor loaded with contrast medium. This simulation was conducted using the electron gamma shower version 4 (EGS4) system as described by Nelson W R, et al. The EGS4 code system Report SLAC-265 Stanford, Calif.: Stanford Linear Accelerator Center, (1985) and the DOSXYZ user code for describing phantom geometries in a Cartesian coordinate system.

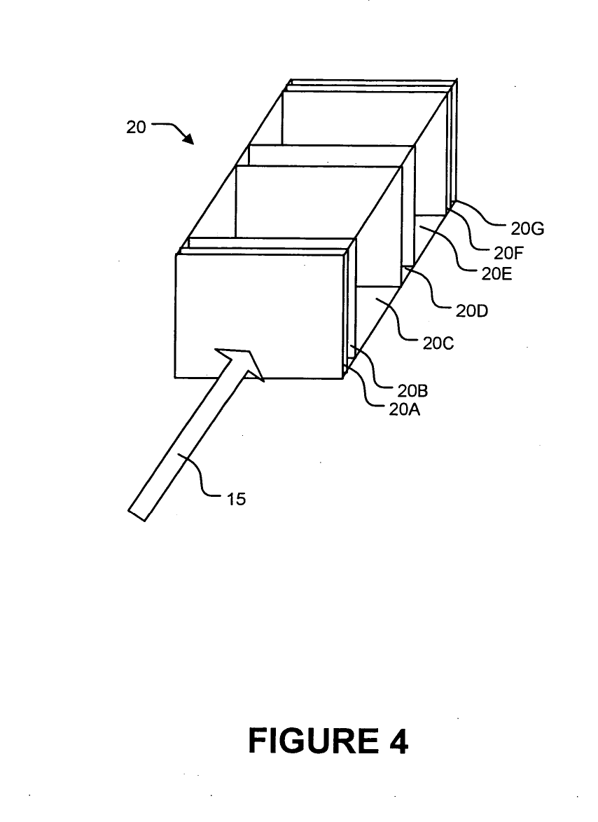

FIG. 4 shows the phantom 20 used in this example. Phantom 20 comprises 7 parallel slabs. X-ray photon beam 15 is perpendicularly incident upon a slab 20A representing skin (0.2 cm thick), a slab 20B representing bone (0.5 cm thick), a slab 20C representing tissue (7.0 cm thick), a central slab 20D representing a tumor containing gadolinium- or iodine-based contrast medium (3.0 cm thick) a slab 20E representing tissue (7.0 cm thick), a slab 20F representing bone (0.5 cm thick), and a slab 20G representing skin (0.2 cm thick). Each slab in the phantom was subdivided into scoring voxels sufficiently small to record the variation of dose along the depth axis of the beam.

For the divergent beams previously modeled using BEAM, the field size was 2.5 cm diameter at isocentre, 100 cm from the source, at the mid-plane of the phantom (9.2 cm depth). All particles recorded in the phase-space file were used, including photons as well as electron or positron contamination.

For the published spectra used, the field size was set to 2.5 cm×2.5 cm at isocentre. The material for the tumor region was defined using the PEGS4 (preprocessor for EGS4) utility, and is composed of a known concentration gadolinium or iodine within water.

Separate simulations were conducted with ratios of (iodine or gadolinium atoms):(water molecules) 40.0 cm of 0, 1:30 000, 1:3000, 1:300, 1:100, 1:30, 1:10 and 1:3. This results in a range of contrast medium concentration from 0 mg/ml (i.e. no contrast medium added) to a maximum that is practically unfeasible (2350 mg/ml and 2910 mg for iodine and gadolinium contrast medium, respectively). This range was set intentionally broad in order to observe the general trend of dose enhancement with tumor contrast medium concentration.

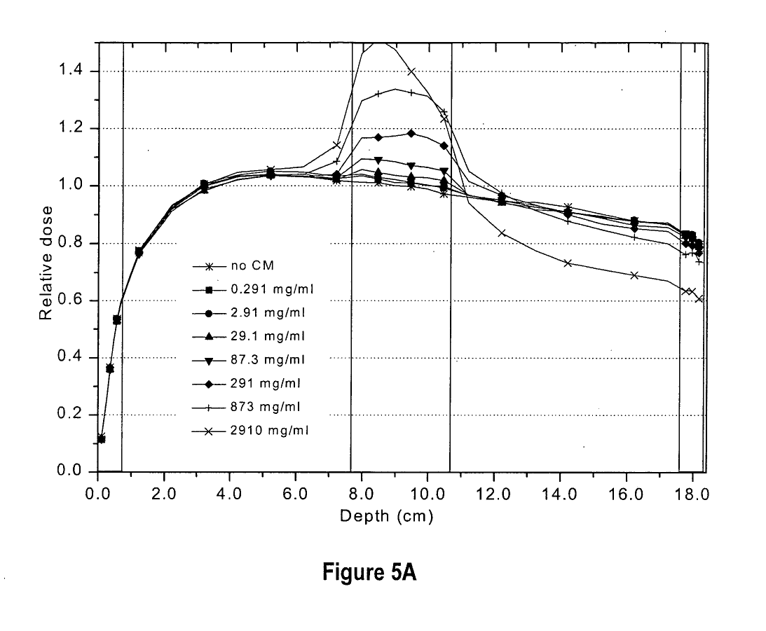

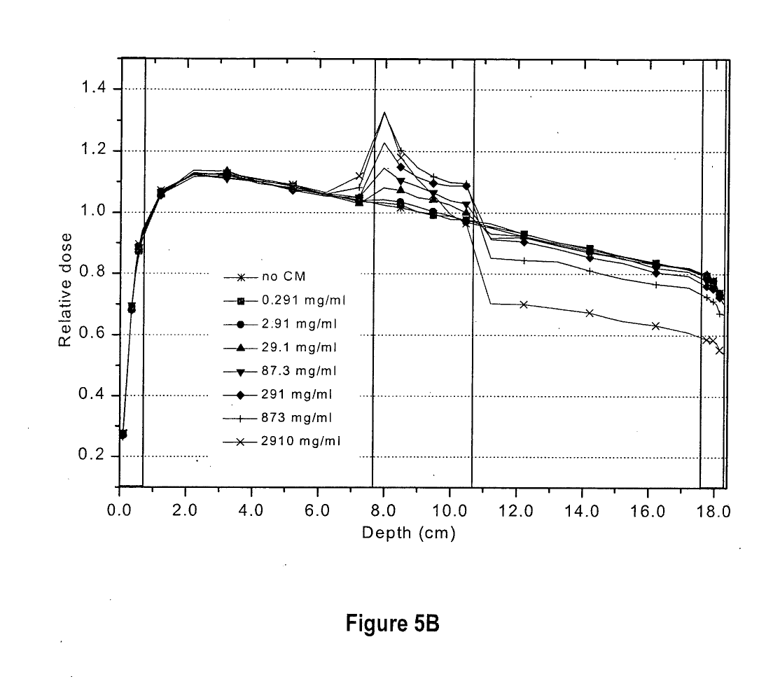

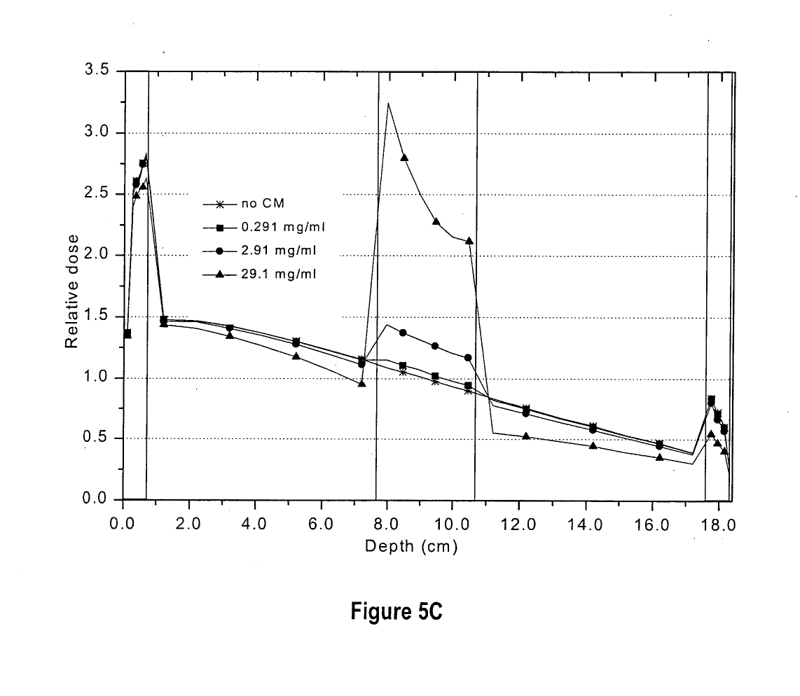

Ionization chamber measurements were performed as a check of the simulation, for one known concentration of iodine contrast medium. FIGS. 5A to 5C show the relative absorbed dose as a function of depth in phantom 20 for 24 MV, 10 MV and 250 kVp beams and a wide range of gadolinium-based contrast medium concentrations. These data correspond to photon spectra generated with the flattening filter in the linear accelerator. For each beam quality, curves have been normalized to the dose at the center of the tumor region for the homogeneous case (i.e. no contrast medium) The vertical lines in these Figures indicate boundaries between the bone, tissue and contrast-medium-containing regions in phantom 20.

It can be seen from FIGS. 5A to 5C that there is some dose enhancement in contrast-medium-containing region 20D but the dose enhancement is low, reaching only 5% for high but potentially achievable concentrations of contrast medium on the order of 20 to 30 mg/ml. The calculated dose enhancement for the 250 kVp beam is comparable to that reported in previous phototherapy studies.

As shown in FIG. 5C, when dose enhancement is achieved by raising the contrast medium concentration to high levels (e.g. to 29.1 mg/ml for the 250 kVp spectrum), attenuation of the beam by the contrast medium itself becomes apparent. This produces a peak in dose at the proximal tissue tumor interface, with rapid reduction deeper within the tumor region. This is consistent with the reports by previous investigators.

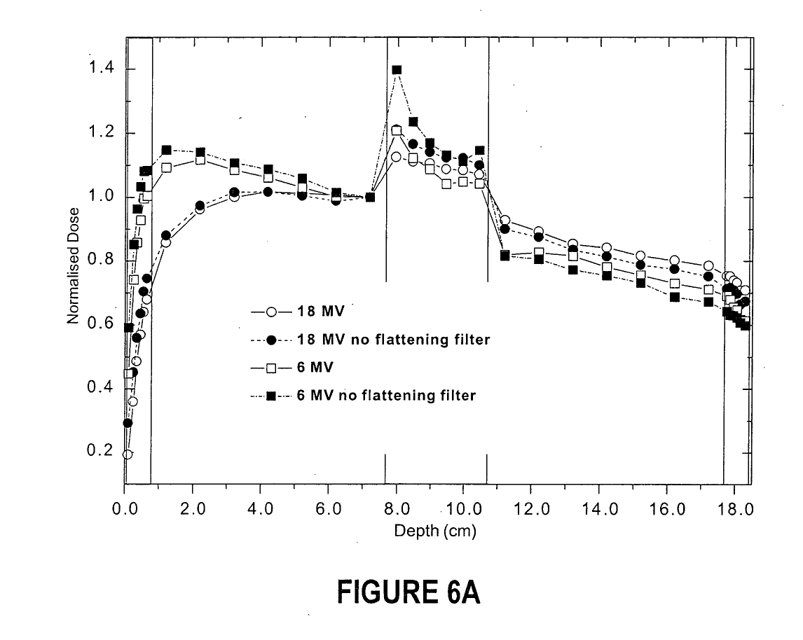

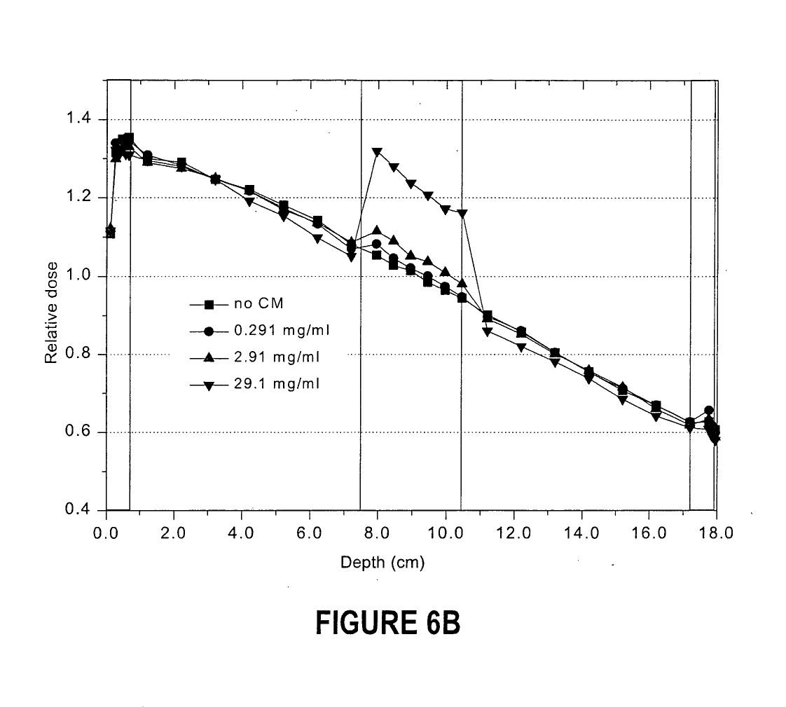

FIG. 6A illustrates the distribution of dose in the simulated phantom for 6 MV and 18 MV beams generated with and without the flattening filter. Removal of the flattening filtration can be seen to increase the dose enhancement significantly.

While the inventors do not wish to be bound by any particular theory of operation it is believed that the reason why removing the flattening filter increases the dose enhancement effect can be understood with reference to FIG. 2. The absence of filtration increases the number of lower energy photons, thus increasing the relative population of photons having energies in the photoelectric absorption region that can interact in the contrast medium-containing tumor via photoelectric absorption. In addition, unlike photon spectra used in phototherapy, the unflattened megavoltage spectra produce buildup at the surface, thereby avoiding significantly enhanced dose in the bone.

FIG. 6B shows the dose enhancement for a 2 MV beam generated without flattening filtration, for contrast medium concentrations that are reasonably achievable within tumors. The combination of lower photon beam quality and absence of flattening filtration produces a significant dose enhancement in the tumor with minimal increase in the dose absorbed within the skull.

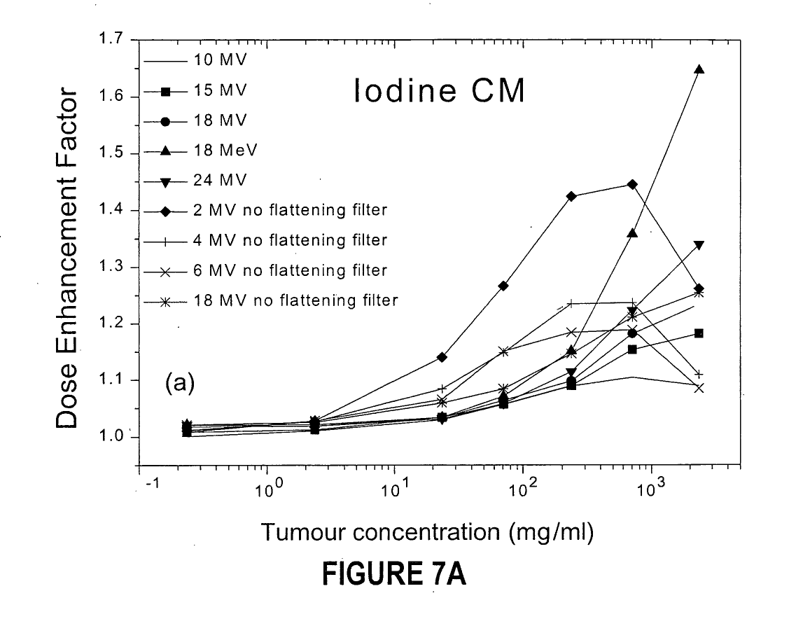

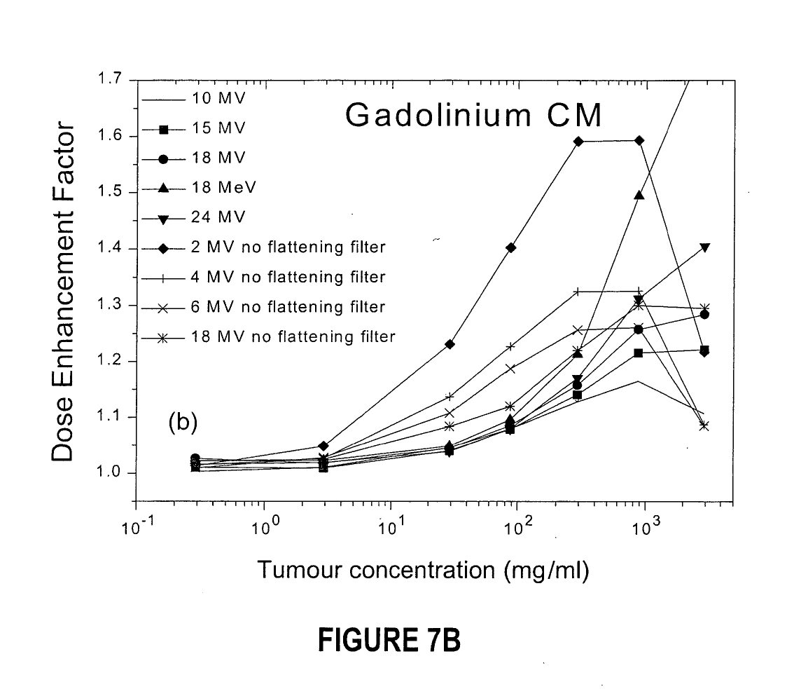

FIGS. 7A and 7B give the dose enhancement factors (DEF) obtained with various beam qualities as a function of contrast medium concentration for iodine- and gadolinium-based contrast media respectively. The DEF is the ratio of the average dose in the tumor region with and without contrast medium. Generally the DEF increases with contrast medium concentration. It can be seen that the rate of increase of DEF with contrast medium concentration is greatest for lower quality photon beams without flattening filtration. For very high concentrations of contrast medium (which are likely unfeasible in practice) DEF actually decreases with concentration due to self-attenuation of the photon beam within the tumor. For the 10, 15, 18 and 24 MV flattened photon spectra, there is no significant variation in DEF with photon beam quality, at least for realistic contrast medium concentrations.

For high-quality photon beams (18 and 24 MV), dose enhancement is evident only for extremely high gadolinium contrast medium concentrations, above 87.3 mg/ml. In this range of contrast medium concentration, the dose enhancement is likely due to the Z² dependence of the pair production atomic cross-section, as evidenced by the fact that the monoenergetic 18 MeV beam yields the highest DEF.

The dose enhancement factors for a contrast medium concentration of 30 mg/ml in the tumor volume are listed in Table 1. Since there was no significant difference in the dose enhancement for flattened beams between 6 MV and 24 MV, mean values are indicated for these beams.

TABLE 1
Dose enhancement factors (DEF) for 30 mg/ml contrast
medium concentration.

| | | Dose enhancement factor (DEF) | |
|---|---|---|---|
| Photon beam | Flattening filter | Iodine CM | Gadolinium CM |
| 10, 15, 18 and | Present | 1.039 | 1.044 |
| 24 MV | | | |
| 18 MeV | N/A | 1.043 | 1.049 |
| 18 MV | Removed | 1.066 | 1.084 |
| 6 MV | Removed | 1.084 | 1.108 |
| 4 MV | Removed | 1.099 | 1.137 |
| 2 MV | Removed | 1.158 | 1.231 |

Table I shows that, with an unflattened 4 MV beam, approximately 14% dose enhancement can be achieved with a gadolinium contrast medium concentration of 30 mg/ml. With an unflattened 2 MV beam increased dose enhancement factors on the order of 16% and 23% can be achieved using 30 mg/ml concentrations of iodine- and gadolinium-based contrast medium, respectively.

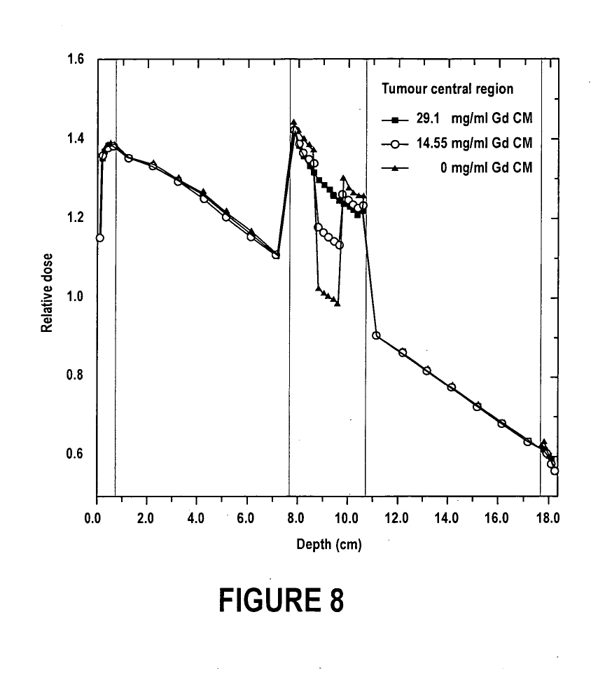

FIG. 8 shows the results of a modeling a case where contrast medium uptake is inhomogeneous. The simulation used to produce the results shown in FIG. 8 included a central 1.0 cm thick region of decreased contrast medium concentration within the 3.0 cm thick treatment volume 20D (FIG. 4). FIG. 8 compares the variation of dose with depth in the simulated phantom using a 2 MV unflattened incident beam, with (i) uniform 29.1 mg/ml gadolinium contrast medium concentration, (ii) a central region containing 14.55 mg/ml gadolinium and (iii) a central region without contrast medium uptake. FIG. 8 demonstrates that the dose enhancement closely matches the spatial distribution of contrast medium, which is consistent with the majority of the dose enhancement being caused by increased absorption of lower energy photons. The photoelectrons thus produced have short ranges in tissue, and thus the boundary between high and low regions of contrast medium concentration is not significantly blurred.

It can be seen from the foregoing that significant dose enhancement can be achieved in treatment volumes containing high atomic number contrast media by irradiating the treatment volume with a megavoltage photon beam having an energy spectrum which provides a significant flux of photons having energies in the photoelectric absorption range lying below 1 MeV and yet a mean energy of at least 1 MeV. Such photon beams tend to spare superficial tissues such as skin and skull.

A suitable beam may be generated using a linear accelerator operating without a flattening filter. The linear accelerator may be operated to so that the energy spectrum has maximum photon energies in the range of 1 MeV to 6 MeV, preferably 1 MeV to 2 MeV. Although these photon spectra contain an increased population of low-energy photons contributing to dose enhancement, the mean photon energy of the incident spectrum remains sufficiently high to produce a build-up effect at the subject's surface. In some embodiments, a majority of energies within the photoelectric absorption range lie below an energy at which the energy spectrum has a maximum fluence.

As shown in FIG. 6B, if a 2 MV beam is used, only a slight increase in the dose to bone is observed. Moreover, this single-beam geometry represents the worst case in terms of the possible increase in the dose to bone.

Photon beams having sufficient uniformity for use in the methods of the invention may be produced from linear accelerators without the requirement of a flattening filter using teletherapy units such as the Cyberknife™ (Accuray Incorporated, Sunnyvale, Calif.) as well as in conventional linear accelerators.

Small diameter beams may be used in some embodiments of this invention to increase beam uniformity in the absence of a flattening filter. For example, in typical current medical linear accelerators 5% dose uniformity can be obtained in beams up to 50 mm diameter without beam flattening. It should be noted that a linear accelerator may need to be modified in order to function acceptably without a flattening filter. In particular, it may be necessary to adjust the beam strength monitoring system to reflect the increase in dose rate achieved through removal of the flattening filter, as well as the beam steering servo system. O'Brien et al. Radiosurgery with unflattened 6-MV photon beams Med. Phys. 18 519-21 (1991) describes modifications made to a particular linear accelerator to permit operating without a flattening filter to increase beam intensity and reduce treatment times. Prior to first treatment, beam re-commissioning is required, including measurements of linear accelerator output, depth doses, relative dose factors and beam uniformity. In addition, quality assurance is necessary after removal and reinstallation of the flattening filter to verify both machine output and servo beam steering.

The application of the invention is not limited to treatment of intracranial lesions. The technique may also be advantageous for very common radiotherapy procedures such as treatment of prostate cancer, for which dose escalation to the tumor volume has been shown to result in improved clinical outcome, yet remains difficult due to prostate motion during treatment and the proximity of dose-limiting structures (i.e. bladder or rectum).

In addition to radiotherapy or radiosurgery of malignant and benign tumors, the methods of the invention may be applied in the treatment of arteriovenous malformation (AVM) or other conditions for which radiotherapy of a treatment volume which can contain a contrast medium can be beneficial. AVMs comprise one of the most frequent indications treated using radiosurgery. The deliverable radiation dose is typically limited by the volume of the AVM itself due to the risk of causing necrosis of surrounding normal tissues. During imaging using angiography, AVMs are very clearly visualized due to the high concentration of contrast medium flowing through the nidus and surrounding vessels. If administered prior to radiosurgical treatment, the presence of a significant amount of contrast medium could yield a high dose enhancement within the AVM itself with sparing of surrounding normal tissues. Compared to treatment of tumors with contrast medium dose enhancement, however, more expedient treatment delivery is required since contrast medium can wash out of vasculature rapidly.

Because most medical linear accelerators are not designed to be used without flattening filters, the invention provides a multimode linear accelerator which has a first mode for operating without a flattening filter to deliver radiation in methods of the invention and one or more second modes for operating with a flattening filter for conventional radiotherapy or radiosurgery.

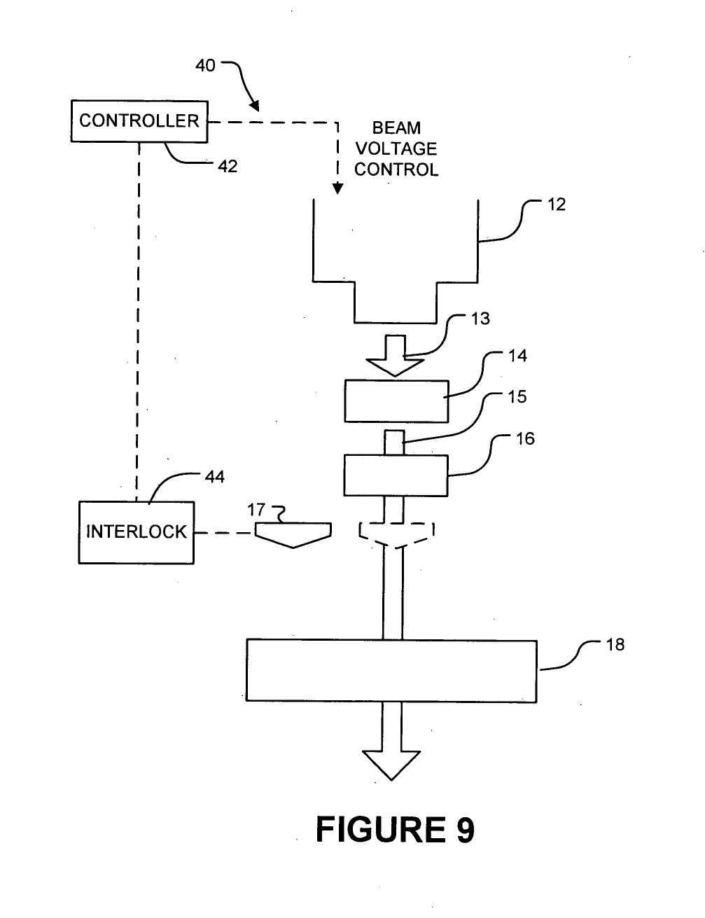

FIG. 9 shows a linear accelerator 40 according to an embodiment of the invention. Components in FIG. 9 are indicated by the same reference numerals used to identify similar components in the prior art linear accelerator of FIG. 1.

Accelerator 40 has a controller 42. Flattening filter 17 can be selectively inserted or removed from the path of photon beam 15. An actuator controlled by controller 42 may be provided to remove or insert flattening filter 17 in the path of photon beam 15. An interlock mechanism 44 is connected to signal to controller 42 when flattening filter 17 is in the photon beam and flattening filter 17 is not in the photon beam. Linear accelerator 40 has a first operating mode into which controller 42 can place linear accelerator 40 when interlock mechanism 44 indicates that flattening filter 17 is not in the path of photon beam 15. Linear accelerator 40 also has a second operating mode into which controller 42 can place linear accelerator 40 when interlock mechanism 44 indicates that flattening filter 17 is in the path of photon beam 15.

When linear accelerator 40 is in the first operating mode, controller 42 causes the particle beam of accelerator 12 to be operated at a reduced voltage in the range of 1 MV to 6 MV, preferably 1 MV to 4 MV, most preferably 1 MV to 2 MV or 2½ MV so that photon beam 15 has an energy spectrum characterized by a maximum photon energy in excess of 1 MeV, a mean photon energy in excess of 250 keV and a substantial flux of photons in the photoelectric absorption range. In some embodiments the mean photon energy exceeds 750 KeV, and in some cases exceeds 1 MeV.

When linear accelerator 40 is in the second operating mode, controller 42 causes the particle beam of accelerator 12 to be operated at a voltage in excess of the reduced voltage, typically at 4 MV or more. When linear accelerator 40 is commissioned, the photon beams produced in both the first and second modes may be characterized. Subsequently switching between the first and second modes may be performed relatively quickly and easily.

Linear accelerator 40 may have multiple flattening filters for use at different operating voltages.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A radiotherapy method comprising irradiating a treatment volume containing a high atomic number contrast medium with a photon beam having an energy spectrum characterized by a maximum photon energy in excess of 1 MeV, and a mean photon energy in excess of 250 keV.

2. A radiotherapy method according to claim 1 wherein the mean photon energy is in excess of 1 MeV.

3. A radiotherapy method according to claim 1 wherein the energy spectrum is characterized by a maximum fluence at an energy in excess of 200 keV.

4. A radiotherapy method according to claim 3 wherein the photon beam contains photons having energies of less than 400 keV which are absorbed by the contrast medium in the treatment volume.

5. A radiotherapy method according to claim 3 comprising producing the photon beam in a linear accelerator.

6. A radiotherapy method according to claim 5 wherein the linear accelerator comprises a target, the photon beam originates at the target and the photon beam encounters no flattening filter between the target and the treatment volume.

7. A radiotherapy method according to claim 6 comprising operating the linear accelerator at a voltage not exceeding 4 MV.

8. A radiotherapy method according to claim 6 comprising operating the linear accelerator at a voltage not exceeding 2.2 MV.

9. A radiotherapy method according to claim 6 comprising operating the linear accelerator at a voltage in the range of 1 MV to 4.5 MV.

10. A radiotherapy method according to claim 3 wherein the photon beam has a diameter not exceeding 60 mm.

11. A radiotherapy method according to claim 10 wherein the photon beam has an intensity which is uniform to within 5%.

12. A radiotherapy method according to claim 3 wherein the contrast medium contains iodine.

13. A radiotherapy method according to claim 3 wherein the contrast medium contains lutetium.

14. A radiotherapy method according to claim 3 wherein the contrast medium contains gadolinium.

15. A radiotherapy method according to claim 3 wherein the contrast medium contains gold.

16. A radiotherapy method according to claim 3 wherein the treatment volume is intracranial.

17. A radiotherapy method according to claim 3 comprising delivering the contrast medium into the treatment volume by way of a catheter extending into the treatment volume.

18. A radiotherapy method according to claim 3 wherein the treatment volume contains tumor tissue.

19. Use of a linear accelerator operating at a voltage in the range of 1 MV to 4.5 MV to generate a photon beam having an energy spectrum characterized by a maximum photon energy in excess of 1 MeV, a mean photon energy in excess of 250 keV, and a maximum fluence at an energy in excess of 100 keV for irradiation of a treatment volume within a subject and containing a high atomic number contrast medium.

20. A linear accelerator for use in radiotherapy, the linear accelerator comprising:
an electron accelerator;
a target disposed to receive a beam of electrons from the electron accelerator to generate a photon beam;
one or more flattening filters selectively insertable into a path of the photon beam;
a controller;
an interlock mechanism connected to signal to the controller when one of the one or more flattening filters is in the photon beam and when none of the one or more flattening filters is in the photon beam;
the linear accelerator having a first operating mode into which the controller can place the linear accelerator when the interlock mechanism indicates that the flattening filter is in the path of the photon beam and a second operating mode into which the controller can place the linear accelerator when the interlock mechanism indicates that one of the one or more flattening filters is in the path of the photon beam;
wherein, when the linear accelerator is in the first operating mode, the controller causes the electron beam to be operated at a reduced voltage in the range of 1 MV to 4 MV to produce the photon beam having an energy spectrum characterized by a maximum photon energy in excess of 1 MeV and a mean photon energy in excess of 250 keV and when the linear accelerator is in the second operating mode the controller causes the electron beam to be operated at a voltage in excess of the reduced voltage.
